(19) United States
(12) Patent Application Publication
(10) Pub. No.: US 2026/0071185 A1
(43) Pub. Date: Mar. 12, 2026

(54) METHODS FOR ENGRAFTING BONE MARROW ORGANOIDS

(71) Applicant: Northwestern University, Evanston, IL (US)

(72) Inventors: Peng Ji, Evanston, IL (US); Ermin Li, Evanston, IL (US); Kehan Ren, Evanston, IL (US)

(21) Appl. No.: 19/321,039

(22) Filed: Sep. 5, 2025

Related U.S. Application Data
(60) Provisional application No. 63/691,853, filed on Sep. 6, 2024.

(51) Int. Cl. C12N 5/077; C12N 5/0789

(52) U.S. Cl. CPC C12N 5/0669; C12N 5/0647; C12N 2500/02; C12N 2501/115; C12N 2501/125; C12N 2501/14; C12N 2501/145; C12N 2501/155; C12N 2501/165; C12N 2501/22; C12N 2501/2303; C12N 2501/2306; C12N 2501/2321; C12N 2501/26; C12N 2503/04; C12N 2506/45

(57) ABSTRACT

The present invention provides methods for engrafting a bone marrow organoid with CD34+ hematopoietic stem and progenitor cells. Compositions comprising the factors needed for engraftment are also provided.

### CROSS-REFERENCE TO RELATED APPLICATIONS

This application claims the benefit of and priority to U.S. Provisional Application No. 63/691,853 filed on Sep. 6, 2024. The content of which is incorporated by reference in its entirety.

### STATEMENT REGARDING FEDERALLY SPONSORED RESEARCH

This invention was made with government support under grant numbers HL169507, DK138205, DK124220, and CA289959 awarded by the National Institutes of Health. The government has certain rights in the invention.

### BACKGROUND

One of the significant challenges in the study of hematologic diseases is to find appropriate model systems that closely recapitulate human pathophysiology in vivo. The development of immune-deficient mouse models, including NSG, NSGS, and more recent MISTG mice, revolutionized the field by facilitating the engraftment of human hematopoietic stem and progenitor cells (HSPCs) in the humanized bone marrow microenvironment in immunodeficient mice. However, it remains a major obstacle to engraft HSPCs from many diseases, such as myelodysplastic syndromes (MDS), into these models. Disparities in immune responses and differences in bone marrow microenvironment between humans and mice often lead to results that may not accurately translate to human conditions.

The human bone marrow organoid is a more suitable model for studying human hematopoiesis on a tissue scale. Prototype bone marrow organoids developed over the past decades applied biomimetics to replicate the three-dimensional architecture of the human bone marrow. They are often supplemented with feeder cells and/or exogenously provided growth factors to recreate the bone marrow microenvironment. Despite these efforts, these models lack the self-sustainability for autonomous hematopoiesis and faithful recapitulation of the bone marrow environment. More recent advancements in the field using human induced pluripotent stem cells (iPSCs)-derived bone marrow organoid models resolve many of these issues by closely mimicking the complex cellular composition and functionality of the native bone marrow. The efficiency of these iPSC-derived organoids in engrafting some of the more challenging HSPCs is of interest.

### SUMMARY

In a first aspect, provided herein is a method for engrafting a bone marrow organoid with CD34+ hematopoietic stem and progenitor cells (HSPCs), the method comprising: (a) incubating the organoid in a cell suspension comprising CD34+ HSPCs in a serum-free stem cell culture medium and a basement membrane matrix; (b) adding to the organoid an engraftment-promoting medium comprising the serum-free stem cell culture medium, stem cell factor (SCF), fms related receptor tyrosine kinase 3 (FLT3), thrombopoietin (TPO), erythropoietin (EPO), and interleukin-3 (IL-3).

The step of incubating the organoid in the cell suspension may be done for between about 15 and about 30 minutes. The step of incubating the organoid in the cell suspension may be done at between about 36.5 and about 37.5° C.

The engraftment-promoting medium may comprise each of SCF, FLT3, TPO, EPO, and IL-3 at between about 5 and about 15 ng/mL. The engraftment-promoting medium may comprise each of SCF, FLT3, TPO, EPO, and IL-3 at about 10 ng/mL.

The cell suspension may comprise between about 1×10³ and about 10×10³ HPSCs. The cell suspension may comprise about 5×10³ HSPCs. The HSPCs may be human HSPCs.

The HSPCs may be isolated from a bone marrow sample.

The method may further comprise preparing the cell suspension before step (a), wherein preparing the cell suspension comprises: suspending the HSPCs in serum-free stem cell culture medium; and adding basement membrane matrix, wherein the basement membrane matrix is at about 4° C.\

Before step (a), the organoid may be matured for between about 18 and about 24 days. The organoid may be matured for about 21 days.

The method may further comprise generating the organoid before step (a), wherein generating the organoid comprises: culturing induced pluripotent stem cells (iPSCs) on the basement membrane matrix; disrupting the cells to form aggregates; plating the aggregates onto an ultra-low attachment substrate to form embryo bodies; contacting the embryo bodies with bone morphogenic protein-4 (BMP4), vascular endothelial growth factor A (VEGFA), fibroblast growth factor-2 (FGF2), and interleukin-21 (IL-21) under hypoxia conditions; culturing the embryo bodies with SCF and FLT3 under normoxia; incubating the embryo bodies in a hydrogel; adding a stem cell differentiation medium supplemented with VEGFA, vascular endothelial growth factor C (VEGFC), FGF2, BMP4, FLT3, SCF, granulocytic colony-stimulating factor (G-CSF), TPO, EPO, IL-3, and interleukin-6 (IL-6) to the hydrogel form organoids; seeding the organoids individually in an ultra-low attachment plate in the stem cell differentiation medium supplemented with FGF2, SCF, FLT3, IL-3, TPO and EPO. The iPSCs may be human iPSCs.

In another aspect, provided herein is a composition comprising a serum-free cell culture medium, SCF, FLT3, TPO, EPO, and IL-3. Each of the SCF, FLT3, TPO, EPO, and IL-3 may be at between about 5 and 15 ng/mL.

In an aspect, provided herein is a kit comprising the composition described herein; and at least one of a CD34+ HSPC and a bone marrow organoid.

In another aspect, provided herein is a kit comprising SCF, FLT3, TPO, EPO, and IL-3 in separate containers. The kit may further comprise at least one of a CD34+ HSPC and a bone marrow organoid.

In another aspect, provided herein is a method for predicting drug response in a subject having a myeloid disease, the method comprising: (a) engrafting a bone marrow organoid with CD34+ HSPC cells isolated from the subject by the method described herein, thereby producing an engrafted organoid; (b) treating the engrafted organoid with the drug; and (c) assessing viability of the CD34+ HSPC cells in the engrafted organoid.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

FIGS. 1A-1F. Human iPSC-derived bone marrow organoid mimics human bone marrow structure. (A) Wide-field picture of a matured human bone marrow organoid in culture on day 21. (B) H&E staining of the bone marrow organoid demonstrating the presence of hematopoietic cells within sinusoids. (C) Representative pictures of transmission electron microscopy reveal diverse cell types within bone marrow organoids. (D) 3D whole-mount imaging of human bone marrow organoids with cell surface rendered via Imaris (right). Organoids were immunostained for CD45+ hematopoietic cells and CD271+ mesenchymal stromal cells, with biotinylated UEA 1 highlighting endothelial cells. (E) 3D imaging reveals erythroblasts, stained for CD235a, encapsulated within UEA1+ sinusoidal vessels. (F) Comparison of confocal images between organoid-derived erythroblasts and those from a human bone marrow core biopsy.

FIGS. 2A-2E. (A-E) Validation of multilineage hematopoiesis within the organoid through flow cytometry. After digestion with collagenase into a single-cell suspension, organoid-derived cells were stained as indicated. Labels in the bracket symbols indicate the cell populations the corresponding panels were based on for analyses. The percentages of the indicated populations are presented.

FIGS. 3A-3C. Genetic analysis of iPSC-derived hematopoietic cells. (A) Uniform manifold approximation and projection (UMAP) plot demonstrating the indicated cell clusters from the single-cell RNA sequencing of human iPSC-derived bone marrow organoid. MSC: mesenchymal stromal cells. (B) Heat map of highly expressed genes in the indicated cell clusters. (C) Cytogenetic analysis of iPSC-derived human bone marrow organoids demonstrates a normal female karyotype.

FIGS. 4A-4C. Human iPSC-derived bone marrow organoid supports autonomous hematopoiesis in humanized immunodeficient mice. (A) Organoid implantation in NSG mice. The organoid was implanted under the renal capsule of the left kidney. H&E staining demonstrates the successful engraftment one-month post-implantation. Red arrows indicate vessel lumens enclosing red blood cells. (B) Flow cytometry analysis of RBC-depleted peripheral blood at 1-, 5-, and 11-month post-implantation to compare the percentage of human and mouse CD45+ leukocytes. CD235a+ human erythroid cells were also tested at 5 months using non-RBC-depleted samples. (C) Quantitative analysis of the ratio of human and mouse CD45+ cells in B. Each data point represents an NSG mouse implanted with a human bone marrow organoid.

FIGS. 5A-5D. Donor HSPCs engraft within bone marrow organoids and recapitulate disease pathophysiology. (A) Confocal imaging demonstrates engraftments of CellVue-labeled donor cells: 5×10³ CellVue-labeled CD34+ HSPCs derived from a normal bone marrow sample were co-incubated with an organoid in a 96-well plate for 3 days before imaging. Arrows indicate CellVue+ cells reside within an erythroid island. (B) Confocal imaging demonstrates engraftments and focal proliferation of CellVue-labeled donor cells: 5×10³ CellVue-labeled CD34+ HSPCs derived from a normal bone marrow sample were co-incubated with an organoid in a 96-well plate for 3 days before imaging. Scale bar: 100 μm. (C) 3D imaging reveals HSPCs from the normal bone marrow samples engraft within the vascular niches of the organoids. (D) Flow cytometry analysis of organoids engrafted with CellVue-labeled HSPCs as in A. 10 organoids were pooled together for each flow cytometry assay. None engrafted organoids were used as controls.

FIGS. 6A-6F. Proliferation and survival analyses of the engrafted CD34+ HSPCs within human bone marrow organoids. (A) Cell Trace proliferation assay of the engrafted CD34+ HSPCs on day 2, day 5, and day 10, respectively. 1×10⁶ CD34+ HSPCs were pooled from three independent MDS samples and stained with Cell Trace Far Red dye. 5×10³ CD34+ stained HSPCs were engrafted into each organoid, at least 10 organoids were pooled for each flowcytometry assay. The Cell Trace intensity of the engrafted CD34+ cells was tested on day 2, day 5, and day 10 post engraftment. (B) Cell death of the engrafted CD34+ cells were assessed using DAPI and quantified in (C). (D) The total output of hematopoietic cells per 1000 CD34+ HSCPs engrafted was assess with cell counting beads. (E) Secondary engraftment of CD34+ HSPCs. CellTrace+ HSPCs were sorted using FACS on day 3 after primary engraftment. Live sorted HSPCs (5×10³) were engrafted into each human bone marrow organoid. The secondary engraftment was assessed with whole-mount 3D imaging. (F) Flow cytometry analysis of the indicated cell lineages in the CellTrace+ engrafted donor cells in the secondary engrafted organoids. The percentages of the indicated populations are presented. APC, antigen-presenting cell; DAPI, 4′,6-diamidino-2-phenylindole.

FIGS. 7A-7C. Donor HSPCs engraftments recapitulate disease pathophysiology. (A) Flow cytometry analyses show multilineage differentiation within organoids of CellVue-labeled donor HSPCs. Each organoid was engrafted with 5×10³ CD34+ HSPCs derived from a normal bone marrow sample. 10 organoids were pooled for each assay. The patient's primary bone marrow sample was analyzed, as shown on the right panel, to confirm that the CD34+ cells do not co-express lineage markers. (B) Same as A, except CD34+ HSPCs derived from 3 different MDS patients were engrafted separately. (C) Statistical analysis of CellVue+ engraftments, with each point representing an independent flow cytometry assay using CD34+ HSPCs from a distinct patient sample. 10 organoids were pooled for each assay. n=3 in each group. Student's t test, *p<0.05, **p<0.01, ***p<0.001.

FIG. 8. Histologic examination of bone marrow biopsies. Bone marrow biopsies from the indicated patients at the time of diagnosis were performed for H&E stains (left) and CD34 immunohistochemical stains (IHC) (right). Large arrows in MDS patients 1 and 2 indicate dyserythropoiesis of irregular nuclear contour. Small arrows indicate binucleated erythroblasts. The arrow in patient 3 indicates a dysplastic megakaryocyte. Stars indicate blasts. Scale bars: 20 μm.

FIG. 9. Complete blood counts of indicated patients over time.

FIGS. 10A-10C. The engraftment-derived hematopoietic cells retain the pathological characteristics both genetically and morphologically. (A) Whole exome DNA sequencing of the engraftment-derived hematopoietic cells from day 10 post engraftment. (B) Prussian blue staining of the bone marrow smear sample from Patient S2. (C) Prussian blue staining of the Cytospin sample from organoids on day 10 post-engraftment with CD34+ HSPCs from Patient S2.

FIGS. 11A-11B. Flow cytometry analysis of engrafted bone marrow organoids. (A) Gating strategy used to identify viable engrafted cells. Cells positive for CellVue Far Red were analyzed for Annexin V and DAPI staining. The population negative for both Annexin V and DAPI was defined as viable. (B) Quantification of viable cells across treatment groups based on the gating strategy shown in (A). Data represent the percentage of Annexin V/DAPI cells among the CellVue population.

### DETAILED DESCRIPTION

The present disclosure provides methods for engrafting a bone marrow organoid with CD34+ hematopoietic stem and progenitor cells (HSPCs). As shown in the examples, the inventors have demonstrated that when the HSPCs are incubated with the organoids, they migrate toward the vasculature niche and initiate multi-lineage hematopoietic differentiation with the organoid environment. Within the organoids, differentiation patterns from MDS patient-derived HSPCs are significantly distinct compared to multilineage hematopoiesis from normal HSPCs, which can be correlated with the clinical manifestations of the disease. Engrafted bone marrow organoids prepared by the methods described herein can be used for various applications in research and clinical settings. Key applications include:

1. Disease Modeling and Mechanistic Studies: The organoid system can model hematopoietic diseases, including Myelodysplastic Syndromes (MDS) and other hematologic malignancies. By engrafting patient-derived HSPCs into the organoids, researchers can study disease progression and the underlying mechanisms in a controlled environment that closely mimics the human bone marrow niche.

2. Drug Screening and Therapeutic Development: This organoid platform offers a high-throughput system for screening potential therapeutic agents. The ability to observe the effects of drugs on patient-derived HSPCs within the organoids allows for the identification of compounds that may be effective in treating specific hematologic conditions. Furthermore, the system can be used to test combination therapies and assess their impact on both normal and diseased HSPCs.

3. Personalized Medicine: By using HSPCs derived from individual patients, this technology enables personalized medicine approaches. Researchers can assess the patient's specific response to different treatments, facilitating the development of tailored therapeutic strategies that are more likely to be effective based on the patient's unique hematopoietic profile.

4. Regenerative Medicine: The bone marrow organoids can be employed in regenerative medicine to generate functional hematopoietic tissues. This application holds the potential for developing new treatments for patients with bone marrow failure or those requiring bone marrow transplantation.

5. Gene Editing and Functional Genomics: The organoid system can be a valuable tool for gene editing studies. Researchers can introduce specific genetic modifications into iPSCs or HSPCs and study the resultant effects on hematopoiesis within the organoid environment. This application is crucial for understanding the functional roles of genes in hematopoietic development and disease.

6. Toxicology and Safety Assessment: The organoids can be utilized for toxicology studies to evaluate the safety of new drugs and compounds. By observing the impact on hematopoietic cells within the organoids, researchers can predict potential hematotoxic effects before proceeding to clinical trials.

In a first aspect, provided herein is a method for engrafting a bone marrow organoid with CD34+ hematopoietic stem and progenitor cells (HSPCs), the method comprising: (a) adding to the organoid a cell suspension comprising CD34+ HSPCs in a serum-free stem cell culture medium and a basement membrane matrix; (b) incubating the organoid in the cell suspension; (c) adding to the organoid an engraftment-promoting medium comprising the serum-free stem cell culture medium, stem cell factor (SCF), fms related receptor tyrosine kinase 3 (FLT3), thrombopoietin (TPO), erythropoietin (EPO), and interleukin-3 (IL-3). The method may further comprise, prior to step (a) removing cell culture medium that the bone marrow organoid may be placed in.

CD34 (i.e. cluster of differentiation 34) is a transmembrane phosphoglycoprotein expressed on hematopoietic stem cells (HSPCs) and other cell types. CD34 is a useful marker for identifying and isolating HSPCs, as it is expressed on most human HSPCs, but is absent on mature blood cells. CD34+ HSPCs are multipotent and can give rise to all cell types in blood.

A “serum-free stem cell culture medium” is a cell culture medium specifically formulated to support the development of hematopoietic cells in culture. In exemplary embodiments, the scrum-free stem cell culture medium is StemPro™-34 medium.

A “basement membrane matrix” is a gelatinous extracellular membrane protein substance that mimics the laminin/collagen IV-rich basement membrane extracellular environment found in many tissues. In exemplary embodiments, the basement membrane matrix is Matrigel.

Stem cell factor (SCF) is a cytokine that binds to the c-KIT receptor (CD117) and plays an important role in hematopoiesis. Fms (feline McDonough scarcoma) related receptor tyrosine kinase 3 (FLT3), also called cluster of differentiation antigen 135 (CD135) is a cytokine receptor expressed on the surface of many hematopoietic progenitor cells. FLT3 signaling is important for the normal development of HSPCs. Thrombopoietin (TPO) is a glycoprotein hormone and hematopoietic cytokine produced by the liver and kidney It regulates the production of platelets. Erythropoietin (EPO) is a glycoprotein cytokine that stimulates red blood cell production in the bone marrow. Interleukin-3 (IL-3), also called colony-stimulating factor (CSF), is a cytokine that induces proliferation and differentiation of early pluripotent stem cells.

The step of incubating the organoid in the cell suspension may be done for between about 15 and about 30 minutes, or any duration or range in between. In exemplary embodiments, the organoid and cell suspension are incubated for about 30 minutes. The organoid and cell suspension may be incubated at between about 36.5 and about 37.5° C., or any temperature or range in between. In exemplary embodiments, the organoid and cell suspension are incubated at about 37° C.

The engraftment-promoting medium may comprise between about 5 and about 15 ng/ml, or any concentration or range in between, of each of SCF, FLT3, TPO, EPO, and IL-3. In exemplary embodiments, the engraftment-promoting medium comprises about 10 ng/ml of each of SCF, FLT3, TPO, EPO, and IL-3.

The cell suspension may comprise between about 1×10³ and about 10×10³ HPSCs, or any number or range in between. In exemplary embodiments, the cell suspension comprises about 5×10³ HSPCs. The CD34+ HSPCs may be human cells.

The CD34+ HSPCs may be isolated from a bone marrow sample. The bone marrow sample may be normal bone marrow or bone marrow from a subject having a myeloid disease, including but not limited to myelodysplastic syndrome (MDS), acute myeloid leukemia (AML), chronic myeloid leukemia (CML), or a myeloproliferative neoplasm (MPN). In exemplary embodiments, the subject may have a myelodysplastic syndrome (MDS). The method may further comprise isolating the HSPCs from a bone marrow sample before performing the engraftment. In exemplary embodiments, the CD34+ HSPCs are isolated using a CD34 MicroBead Kit.

Myelodysplastic syndromes are a group of disorders which immature blood cells in the bone marrow (i.e., blasts) fail to mature into healthy blood cells. As a result, the bone marrow produces too few mature, functional red blood cells, white blood cells, or platelets. Symptoms of MDS include fatigue, shortness of breath, bleeding disorders, anemia, and frequent infections. Risk factors for MDS include previous chemotherapy or radiation therapy, exposure to certain chemicals (e.g., tobacco smoke, pesticides, benzene), and exposure to heavy metals (e.g., mercury, lead). CD34+ cells from MDS patients harbor genetic abnormalities that are not found in CD34+ cells from healthy individuals.

There are several different types of MDS. For example, the World Health Organization (WHO) recognizes 6 main types of MDS: MDS with multilineage dysplasia (MDS-MLD), MDS with single lineage dysplasia (MDS-SLD), MDS with ring sideroblasts (MDS-RS), MDS with excess blasts (MDS-EB), MDS with isolated del (5q), and MDS, unclassifiable (MDS-U). Some types of MDS, referred to as “low-risk MDS,” progress slowly and may cause mild to moderate anemia (i.e., a low number of red cells) or decrements to other types of cells. Other types of MDS, referred to as “high-risk MDS” (e.g., MDS-EB), can cause severe problems. In patients with high-risk MDS, immature blood cells referred to as “blasts” make up more than 5 percent of the cells in the bone marrow. The excess blasts do not develop into normal blood cells, which causes severe deficits. Specifically, low blood cell counts can lead to anemia (i.e., low red cell count), neutropenia (i.e., low neutrophil count) or thrombocytopenia (i.e., low platelet count).

Myeloproliferative neoplasms (MPNs) are a group of rare blood cancers in which excess red blood cells, white blood cells or platelets are produced in the bone marrow. MPNs include chronic myeloid leukemia, chronic neutrophilic leukemia, polycythemia vera, primary myelofibrosis, etc. MPNs such as primary myelofibrosis may accelerate and turn into acute myeloid leukemia. Acute myeloid leukemia (AML) is a cancer of the myeloid line of blood cells, characterized by the rapid growth of abnormal cells that build up in the bone marrow and blood and interfere with normal blood cell production. Chronic myeloid leukemia (CML) is a cancer of the white blood cells, characterized by the increased and unregulated growth of myeloid cells in the bone marrow and the accumulation of these cells in the blood. It is a clonal bone marrow stem cell disorder in which a proliferation of mature granulocytes (neutrophils, eosinophils and basophils) and their precursors is found.

The method may further comprise preparing the cell suspension. The cell suspension may be prepared by suspending the CD34+ HSPCs in the serum-free stem cell culture medium, then adding ice-cold basement membrane matrix. The basement membrane matrix may be at a temperature of about 4° C. The volume of basement membrane matrix added may be about half the volume of serum-free stem cell culture medium added. In exemplary embodiments, the CD34+ HSPCs are suspended in about 200 μL serum-free stem cell culture medium, and about 100 μL basement membrane matrix is added.

The bone marrow organoid may be derived from human cells. The organoid may be matured for between about 18 and about 24 days at the beginning of the engraftment method. In exemplary embodiments, the organoid is matured for about 21 days.

The method may further comprise generating the organoid before initiating the engraftment method. Generating the organoid may comprise: culturing induced pluripotent stem cells (iPSCs) on the basement membrane matrix; disrupting the cells to form aggregates/clumps; plating the aggregates onto an ultra-low attachment substrate to form embryo bodies; contacting the embryo bodies with bone morphogenic protein-4 (BMP4), vascular endothelial growth factor A (VEGFA), fibroblast growth factor-2 (FGF2), and interleuin-21 (IL-21) under hypoxia conditions; culturing the embryo bodies with SCF and FLT3 under normoxia; incubating the embryo bodies in a hydrogel; adding a stem cell differentiation medium supplemented with VEGFA, vascular endothelial growth factor C (VEGFC), FGF2, BMP4, FLT3, SCF, granulocytic colony-stimulating factor (G-CSF), TPO, EPO, IL-3, and interleukin-6 (IL-6) to the hydrogel to form organoids; seeding the organoids individually in an ultra-low attachment plate in the stem cell differentiation medium supplemented with FGF2, SCF, FLT3, IL-3, TPO and EPO. The iPSCs may be human iPSCs. Methods for generating bone marrow organoids are further described in International Publication No. WO2023156774, which is incorporated herein by reference in its entirety. The methods described herein are distinguished by the addition of IL-21 and the 1% 02 hypoxia conditions.

The step of culturing the iPSCs may be done for at least 7 days, with passages performed when the cells reach confluence. Before disrupting the cells into aggregates, the method may comprise removing iPSC cells that have self-differentiated. In exemplary embodiments, disrupting the iPSCs to form aggregates comprises adding an enzyme-free human pluripotent stem cell selection and passaging reagent (e.g. ReLeSR™) and manually breaking the culture. After forming the aggregates, clumps larger than 100 μM may be filtered out. The remaining aggregates may be centrifuged and resuspended in a cGMP, feeder-free maintenance medium for human iPS cells (e.g. mTeSR™1) supplemented with 1× rho-kinase (ROCK) inhibitor.

The step of plating the aggregates onto an ultra-low attachment substrate may be done for about 24 hours to form the embryo bodies. Embryo bodies are three-dimensional aggregates formed by pluripotent stem cells. Embryo bodies comprise the three embryonic germ layers and mimic characteristics seen in early-stage embryos.

During the step of contacting the embryo bodies with BMP4, VEGFA, FGF2, and IL-21, the BMP4, VEGFA, and FGF2 may each be provided at about 25 ng/ml and the IL-21 may be provided at about 5 ng/mL. Hypoxia conditions are low oxygen environments that mimic the in vivo conditions of cells, which may range from 1-15% oxygen. In exemplary embodiments, the hypoxia condition is about 1% O2. This step may be performed for about 72 hours.

During the step of culturing the embryo bodies with SCF and FLT3, the SCF and FLT3 may be provided at about 25 ng/mL. Normoxia conditions are normal atmosphere conditions (e.g. about 21% oxygen). This step may be performed for about 48 hours.

In exemplary embodiments, during the step of culturing the embryo bodies in hydrogel, the hydrogel is composed of Geltrex™ (a basement membrane extract containing laminin, collagen IV, entactin, and heparin sulfate proteoglycans), VitroCol® (a human collagen), and Collagen IV. This step may be performed for about 2 hours in an incubator.

A stem cell differentiation medium is then added to the hydrogel, wherein the stem cell differentiation medium is supplemented with VEGFC, FGF2, BMP4, FLT3, SCF, G-CSF, TPO, EPO, IL-3, and interleukin-6 (IL-6) to form organoids. In exemplary embodiments, the stem cell differentiation medium is APEL™2 medium. The VEGFA, VEGFC, FGF2, BMP4, FLT3, SCF, G-CSF, TPO, and EPO may be provided at about 50 ng/ml; and the IL-3, and IL-6 may be provided at about 20 ng/mL. This step may be performed for about 7 days.

After seeding the organoids in the ultra-low attachment plates, the method may further comprise allowing the organoids to mature until day 21 before initiating the engraftment method.

A “subject” refers to a human or non-human mammal, e.g. cows, horses, sheep, pigs, goats, rabbits, dogs, cats, bats, mice, and rats. In preferred embodiments, the subject is a human.

In a second aspect, provided herein in a composition comprising a serum-free cell culture medium supplemented with SCF, FLT3, TPO, EPO, and IL-3. The SCF, FLT3, TPO, EPO, and IL-3 may each be at between about 5 and about 15 ng/ml, or any concentration or range in between. In preferred embodiments, the SCF, FLT3, TPO, EPO, and IL-3 are each at about 10 ng/mL.

In a third aspect, provided herein is a kit comprising SCF, FLT3, TPO, EPO, and IL-3. Each of SCF, FLT3, TPO, EPO, and IL-3 may be provided in a solution or buffer and packaged separately or in separate containers.

In an embodiment, the kit may comprise the composition comprising the serum-free cell culture medium with SCF, FLT3, TPO, EPO, and IL-3 described above; and at least one of a CD34+ HSPC and a bone marrow organoid. The composition, CD34+ HSPC, and bone marrow organoid may be packaged separately.

The kits described herein may further comprise any of the reagents and buffers needed to perform the methods described herein. The kits may further comprise instructions for performing the methods described herein.

Any of the cytokines and proteins described herein, e.g. SCF, FLT3, TPO, EPO, IL-3 VEGFA, VEGFC, FGF2, BMP4, IL-21, IL-6, etc. may be recombinant proteins.

In a fourth aspect, provided herein is a method for predicting drug response in a subject having a myeloid disease, the method comprising: (a) engrafting a bone marrow organoid with CD34+ HSPC cells isolated from the subject by the method described herein, thereby producing an engrafted organoid; (b) treating the engrafted organoid with the drug; and (c) assessing viability of the CD34+ HSPC cells in the engrafted organoid. Step (a) may be done for between about 24 and about 72 hours. In embodiments, step (a) is done for about 48 hours. Step (b) may be done for between about 24 and about 72 hours. In embodiments, step (b) is done for about 48 hours.

In exemplary embodiments, the drug comprises azacitidine and venetoclax. The azacitidine may be provided at about 1 μM. The venetoclax may be provided at about 200 nM.

The method may further comprising engrafting a control bone marrow organoid with CD34+ HSPC cells isolated from the subject by the method described herein; treating the control engrafted organoid with a vehicle, e.g. DMSO; assessing viability of the CD34+ HSPC cells in the control engrafted organoid. The vehicle is a substance that does not impact viability of the cells. The vehicle may be the substance that the drug of step (b) is provided in. The method may further comprise comparing the viability of the CD34+ HSPC cells in the engrafted organoid with the viability of the CD34+ HSPC cells in the control engrafted organoid.

The steps of assessing viability of the CD34+ HSPC cells in the engrafted and/or control engrafted organoid may comprise disassociating the organoid in collagenase at between about 35 and about 40° C. for between about 30 and about 90 minutes, and performing flow cytometry analysis. In embodiments, the organoid may be disassociated in collagenase at about 37° C. for about 60 minutes. To perform flow cytometry analysis, the organoid may be stained with a dye that indicates cell viability, e.g. FITC-conjugated Annexin V.

The myeloid disease may be myelodysplastic syndrome (MDS), acute myeloid leukemia (AML), chronic myeloid leukemia (CML), or a myeloproliferative neoplasm (MPN). In embodiments, the myeloid disease is AML.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of”′ should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The cited references are incorporated by reference herein in their entireties. In the event that there is an inconsistency between a definition of a term in the specification as compared to a definition of the term in a cited reference, the term should be interpreted based on the definition in the specification.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1

### Introduction

One of the significant challenges in the study of hematologic diseases is to find appropriate model systems that closely recapitulate human pathophysiology in vivo. The development of immune-deficient mouse models, including NSG, NSGS, and more recent MISTG mice, revolutionized the field by facilitating the engraftment of human hematopoietic stem and progenitor cells (HSPCs) in the humanized bone marrow microenvironment in immunodeficient mice¹⁻⁵. However, it remains a major obstacle to engrafting HSPCs from many diseases, such as myelodysplastic syndromes (MDS), into these models⁶⁻⁹. Disparities in immune responses and differences in bone marrow microenvironment between humans and mice often lead to results that may not accurately translate to human conditions.

The human bone marrow organoid is a more suitable model for studying human hematopoiesis on a tissue scale. Prototype bone marrow organoids developed over the past decades applied biomimetics to replicate the three-dimensional architecture of the human bone marrow^{10,11}. They are often supplemented with feeder cells and/or exogenously provided growth factors to recreate the bone marrow microenvironment¹²⁻¹⁴. Despite these efforts, these models lack the self-sustainability for autonomous hematopoiesis and faithful recapitulation of the bone marrow environment. More recent advancements in the field using human induced pluripotent stem cells (iPSCs)-derived bone marrow organoid models resolve many of these issues by closely mimicking the complex cellular composition and functionality of the native bone marrow^{15,16}. However, the efficiency of these iPSC-derived organoids in engrafting some of the more challenging HSPCs has not been explored.

In this study, we developed an iPSC-derived human bone marrow organoid model capable of autonomous hematopoiesis and sustaining the bone marrow microenvironment. This system also enables efficient engraftment of HSPCs from patients with MDS into the bone marrow organoids and mirrors the pathophysiology of the disease.

### Methods

### Study Design

Human bone marrow organoids were generated from induced pluripotent stem cells (iPSCs) over 21 days. iPSCs were initially identified and separated from differentiated colonies, then cultured to form embryo bodies in ultra-low attachment dishes. These bodies were exposed to hypoxic conditions with growth factors to induce mesoderm formation and angiogenesis, followed by hemogenic endothelium induction. Embedding in hydrogel created 3D organoids, which were cultured with various factors for 7 days to promote angiogenesis and hematopoiesis. After 14 days, organoids were transferred to 96-well plates for further maturation with specific growth factors to support bone marrow development. The bone marrow organoids were then subjected to imaging and flow cytometry assays as previously described¹⁷⁻²¹. Detailed methods are in the supplemental materials.

Development of iPSC-Derived Human Bone Marrow Organoids

The human induced pluripotent stem cells (iPSCs) were purchased from StemCell Technologies (SCTi003-A). The iPSCs were cultured in a 6-well plate coated with Matrigel (Corning) in mTeSR Plus medium and passaged every other day. Before bone marrow organoid differentiation, the self-differentiated iPSC colonies were removed with pipette tips under a microscope. The remaining iPSCs were detached with ReLeSR and broken into cell clumps with gentle pipetting. The larger clumps were excluded using a 100 μm cell strainer (Corning). The smaller cell clumps were centrifugated and resuspended in mTeSR Plus medium supplemented with 1× ROCK inhibitor (RevitaCell, Gibco). The cell clumps were then transferred to an ultra-low attachment (ULA) 6-well plate to form embryo bodies for 24 hours. The embryo bodies were further stimulated with BMP4, VEGFA, and FGF2 at 25 ng/mL and IL-21 ligand at 5 ng/ml under hypoxia condition (1% O2) for 72 hours for mesoderm formation and angiogenesis induction. For hemogenic endothelium inductions, the embryo bodies were further simulated under normoxia for 2 days with the addition of SCF and FLT3 at 25 ng/ml. Subsequently, the embryo bodies were embedded in hydrogel composed of Geltrex (Gibco), VitroCol (Advanced Biomatrix), and Collagen IV (Advanced Biomatrix) in a 24-well plate. After 2 hours of solidification of the hydrogel in an incubator, the APEL2 medium supplemented with VEGFA, VEGFC, FGF2, BMP4, FLT3, SCF, G-CSF, TPO, EPO at 50 ng/mL, IL-3, and IL-6 at 20 ng/mL is added to the hydrogel. After a 7-day hydrogel culture that allows the embryo bodies to mature into self-assembling 3D structured organoids, the organoids were released from the matrix and individually seeded in a 96-well ULA plate in APEL2 medium supplemented with FGF2, SCF, FLT3, IL-3, TPO and EPO 20 ng/ml until day 21, where the step of detaching the iPSCs with ReLeSR and breaking them into clumps with gentle pipetting is day 0.

### Engraftment of Patient-Derived CD34+ HSPCs

CD34+ HSPCs were isolated from bone marrow aspiration samples under an IRB-approved protocol using the CD34 MicroBead Kit (Miltenyi Biotec), according to the manufacturer's instructions, and stored in liquid nitrogen. For engraftment, HSPCs were thawed, labeled with CellVue Claret Far Red Membrane Label (Sigma-Aldrich) as per the manufacturer's guidelines, and resuspended in 200 μL StemPro-34™-medium (Gibco). To this suspension, 100 μL of ice-cold Matrigel (Corning) was added. The medium was removed from day-21 matured organoids, and 30 μL of the cell suspension was applied directly to immerse the organoids, followed by a 30-minute incubation at 37° C. Subsequently, 150 μL of StemPro-34™ medium containing SCF, FLT3, TPO, EPO, and IL-3 (10 ng/mL each) was added to promote engraftment. Engrafted organoids were analyzed via flow cytometry or imaging 3 days post-engraftment.

### Human Bone Marrow Organoid Imaging

Mature organoids were fixed in 2.5% glutaraldehyde in 0.1M phosphate buffer for 72 hours for transmission electron microscopy (TEM) imaging. The transmission electron microscopy was performed by the Center for Advanced Microscopy at Northwestern University.

For 3D confocal microscopy, the organoids were fixed in 4% PFA at room temperature for one hour, permeabilized, and blocked with 0.25% Triton X-100 in 10% goat serum (Thermo Fisher, #50197Z) on an orbital shaker for 2 hours. The organoids were then dehydrated in saturated sucrose at 4° C. for 24 hours before immunostaining with primary and secondary antibodies. After staining, the organoids were immersed in 60% (v/v) glycerol and 2.5 M fructose for 1 hour for tissue clearing. For imaging, organoids were placed in glass bottom dishes (Nunc) and submerged in the clearing buffer for confocal Z-stack imaging. Imaging was conducted using a Nikon AXR laser scanning confocal system with a 20× water immersion objective (CFI Apo LWD Lambda S 20XC WI), capturing Z-steps at 0.85 μm intervals across a total Z distance of at least 120 μm. Z-stack images were processed using Imaris 10.0 (Oxford Instruments) for cell surface rendering.

### Human Bone Marrow Organoid Flow Cytometry

Organoids with or without engrafted patient-derived HSPCs were collected for flow cytometry. Each assay consisted of 10 organoids, which were pooled in MACs buffer (2 mM EDTA in PBS with 0.5% BSA) within 15 mL centrifuge tubes. Following 2 washes in MACs buffer, organoids underwent collagenase digestion using 0.25% collagenase (StemCell Technologies) at 37° C. for 15 minutes to facilitate disassociation into single-cell suspensions. This was achieved through gentle pipetting every 5 minutes during the digestion and subsequent filtration through a 40 μm cell strainer. The resulting single-cell suspensions were centrifuged, resuspended in 200 μL of MACs buffer, and stained with fluorophore-conjugated primary antibodies at a 1:200 dilution. Stained cells were analyzed on a BD Symphony A1 flow cytometer, with data processing and cell population gating conducted in FlowJo, using isotype control antibodies to define gating strategies.

### Cytogenetic Analysis

Conventional cytogenetic analyses of organoid-derived cells were performed using the G-banding method. Following 2 washes in MACs buffer, organoids underwent collagenase digestion using 0.25% collagenase (StemCell Technologies) at 37° C. for 15 minutes to facilitate disassociation into single-cell suspensions for downstream analysis. The fresh cells were harvested from 24-hour unstimulated cultures following standard protocol, which included incubation with colcemid, followed by treatment with hypotonic solution and cell preservation in Carnoy's fixative. Harvested cell suspensions were dropped on to slides and were stained for G-banding with a trypsin-Giemsa solution. The karyotype was described according to the International System for Human Cytogenetic Nomenclature (ISCN 2020).

### Results and Discussion

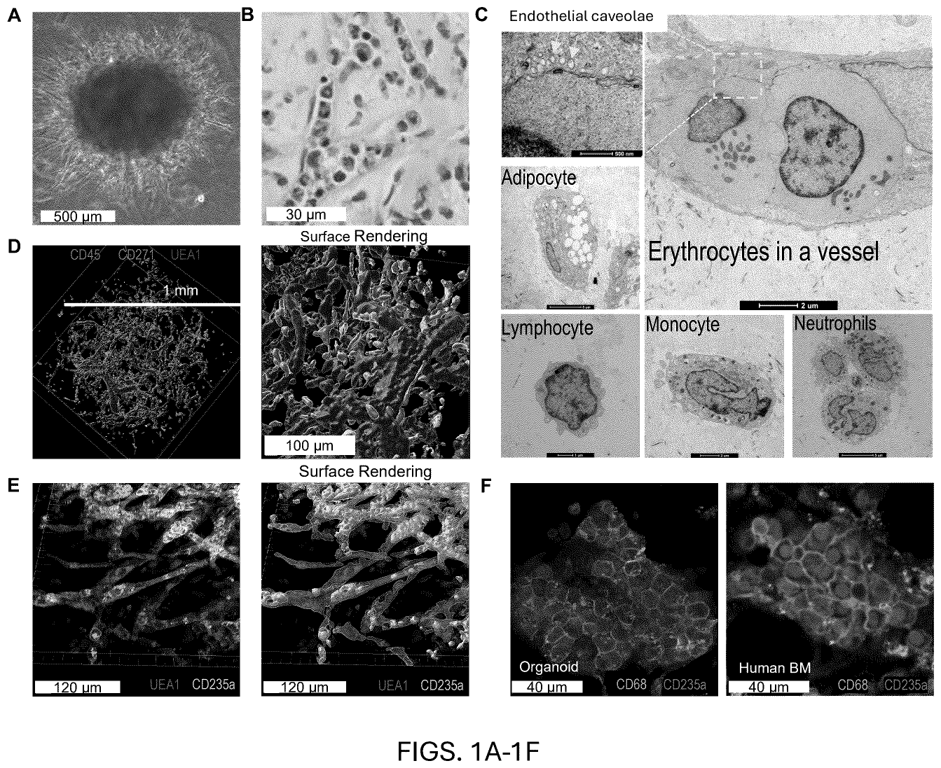

To model human hematopoiesis and the bone marrow microenvironment, we used human iPSCs and differentiated them into bone marrow organoids over 21 days (see methods). By day 21, these organoids form dense spheres around 1,000 μm in diameter (FIG. 1A). Histological analysis confirmed their similarity to human bone marrow with hematopoietic cells inside the microvasculature (FIG. 1B). This was further confirmed by a transmission electron microscopy (TEM) study in which hematopoietic cells, such as erythrocytes, are readily detected in the vessel. Other bone marrow components, including endothelial cells, neutrophils, monocytes, lymphocytes, and adipocytes, were also detected (FIG. 1C). Using whole-mount imaging, we observed a three-dimensional endothelial network interconnected with stromal and hematopoietic cells (FIG. 1D). Cells of the specific lineage, such as erythroid cells, are located adjacent and within the sinusoidal vessels (FIG. 1E). Many erythroblasts also form tight erythroid islands with features consistent with those found in primary human marrow core biopsies (FIG. 1F).

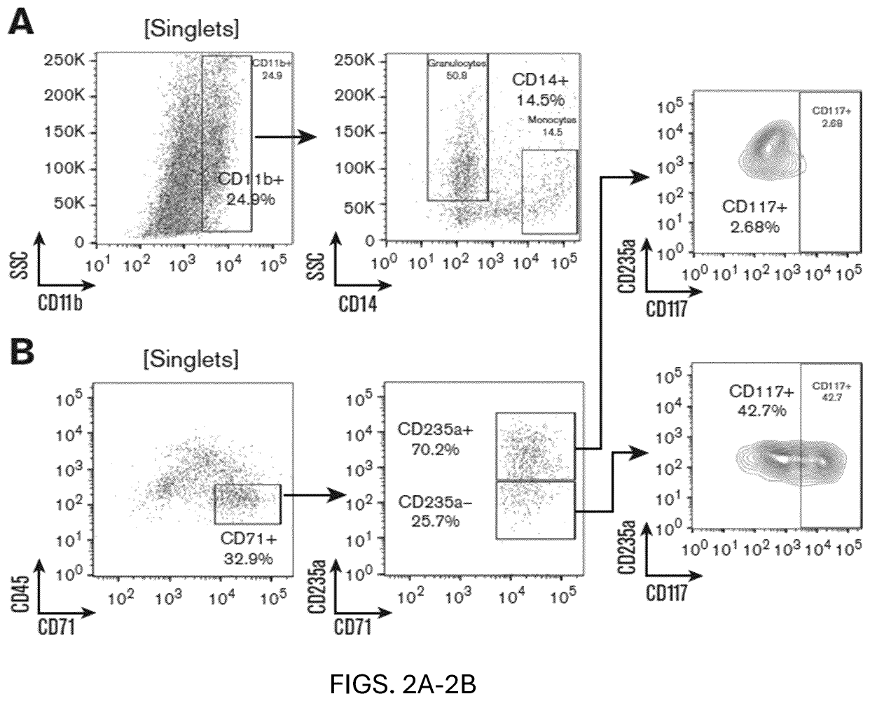

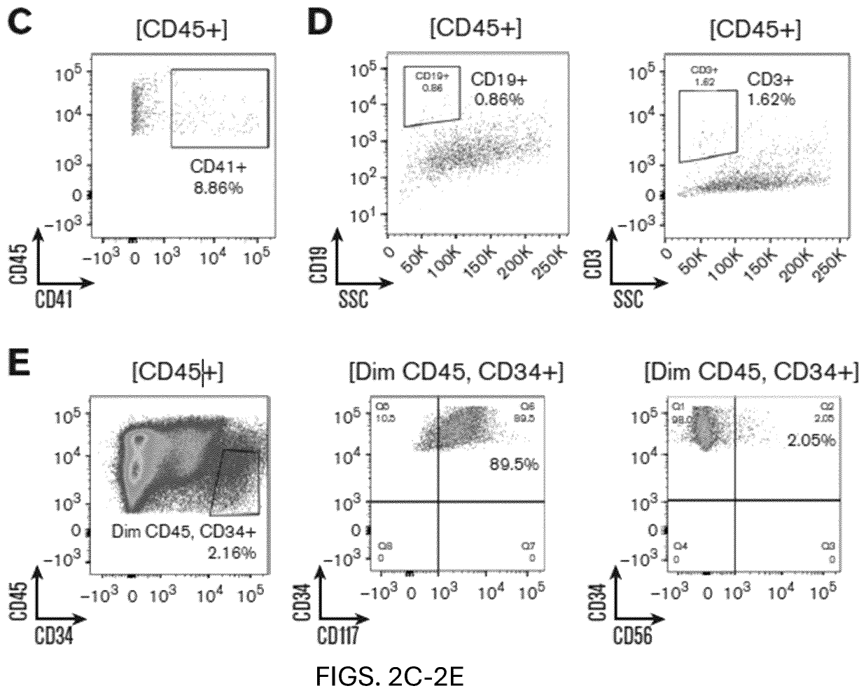

We next performed flow cytometry assays, which validated multi-lineage hematopoiesis and demonstrated that the organoids closely replicate human bone marrow composition, including granulocytes, monocytes, erythrocytes, lymphocytes, and HSPCs (FIG. 2). Organoid-derived myeloid cells expressed CD11b and CD14 in myeloid cells, including monocytes (FIG. 2A). The erythroid cells exhibited mixed stages of differentiation. Early erythrocytes (CD71+ and CD235a−) showed higher expression of progenitor cell marker CD117, which was predominantly negative in CD71+ and CD235a+ late-stage erythroblasts (FIG. 2B). CD41+ megakaryocytic population was also readily detectable (FIG. 2C). However, the lymphoid population remained ambiguous by flow cytometry (FIG. 2D), likely due to the absence of peripheral lymphoid tissues. The organoid-derived CD34+ HSPCs exhibited a normal immunophenotype, expressing CD117 and lacking CD56 expression (FIG. 2E). The composition of the organoids was further investigated using single-cell RNA sequencing. The uniform manifold approximation and projection (UMAP) plot revealed hematopoietic lineages at different stages of differentiation, as well as mesenchymal stromal cells, endothelial cells, and fibroblasts. Importantly, the organoid maintained a normal karyotype, demonstrating its genomic integrity during the differentiation process.

To determine whether bone marrow organoids maintain their properties and functions in vivo over the long term, we implanted them into the renal capsules of immunodeficient NSG mice. The renal capsule provides a suitable microenvironment that supports vascularization, accessibility, immunodeficiency, and growth space necessary for the long-term survival and study of implanted bone marrow organoids in vivo.²⁴

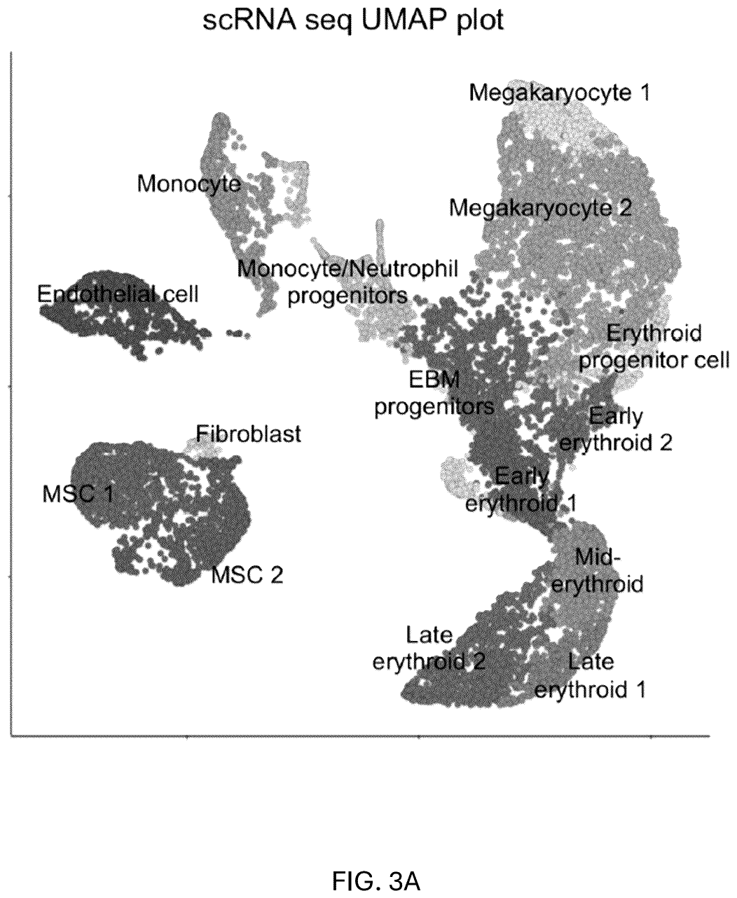

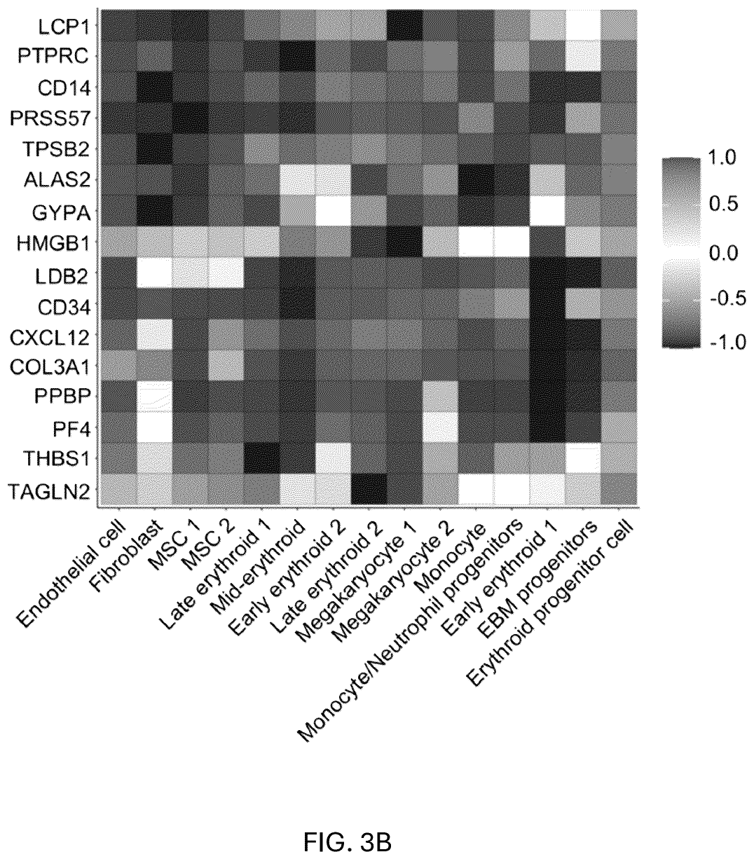

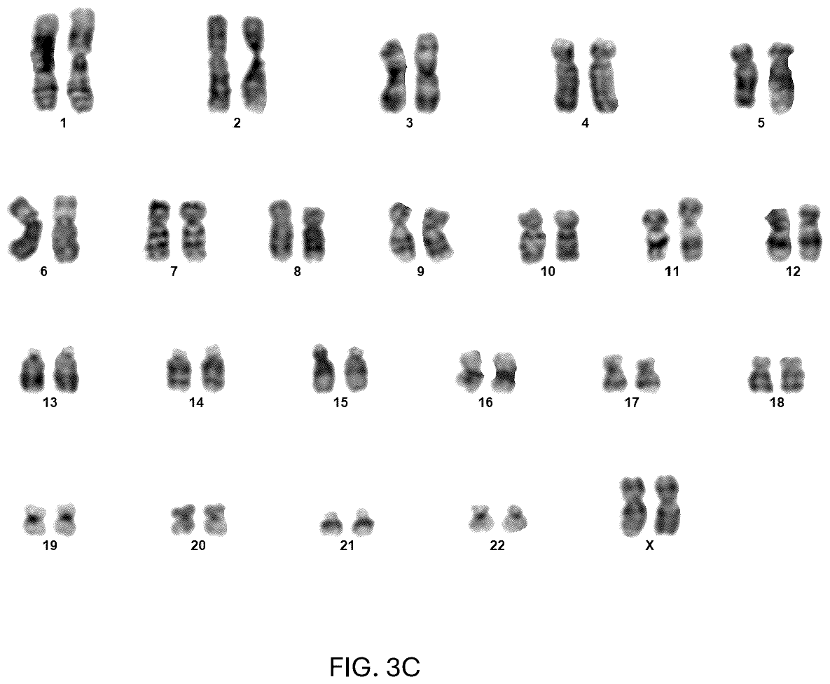

The composition of the organoids was further investigated using single-cell RNA sequencing (FIG. 3A). The uniform manifold approximation and projection (UMAP) plot revealed hematopoietic lineages at different stages of differentiation, as well as mesenchymal stromal cells, endothelial cells, and fibroblasts (FIGS. 3A-3B). Importantly, the organoid maintained a normal karyotype, demonstrating its genomic integrity during the differentiation process (FIG. 3C).

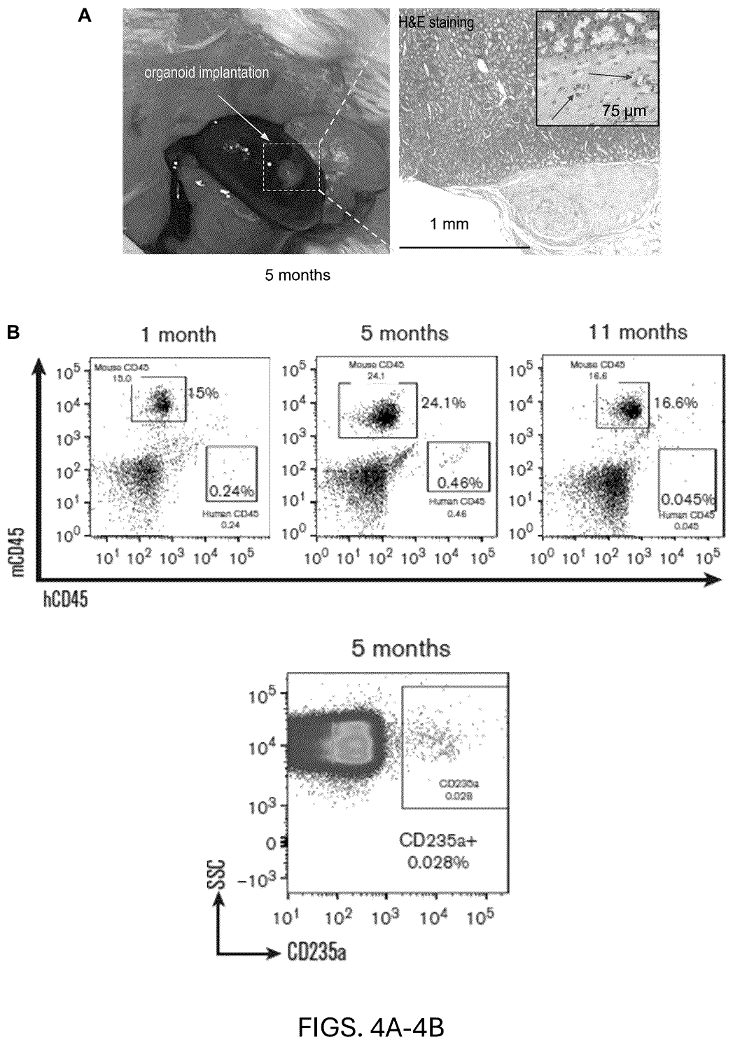

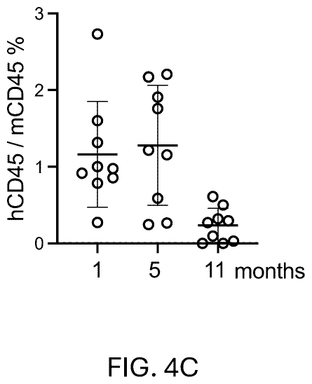

To determine whether bone marrow organoids maintain their properties and functions in vivo in the long term, we implanted them into the renal capsules of the immunodeficient NOD-scidIL2Rγ^{null} (NSG) mice. Gross examination and histologic analysis one month later confirmed successful implantation (FIG. 4A). Human CD45+ leukocytes and CD235a+ erythrocytes were readily identified over 11 months by flow cytometry, albeit with reduced frequency over time (FIG. 4B).

One significant obstacle to hematologic translational studies is the lack of appropriate human models. This is especially problematic in hematologic diseases such as myelodysplastic syndromes (MDS), where HSPCs from these patients are challenging to engraft in immunodeficient mice. This highly replicable and high-fidelity bone marrow organoid system prompted us to investigate whether HSPCs from patients with various forms of MDS could be effectively engrafted into the organoids.

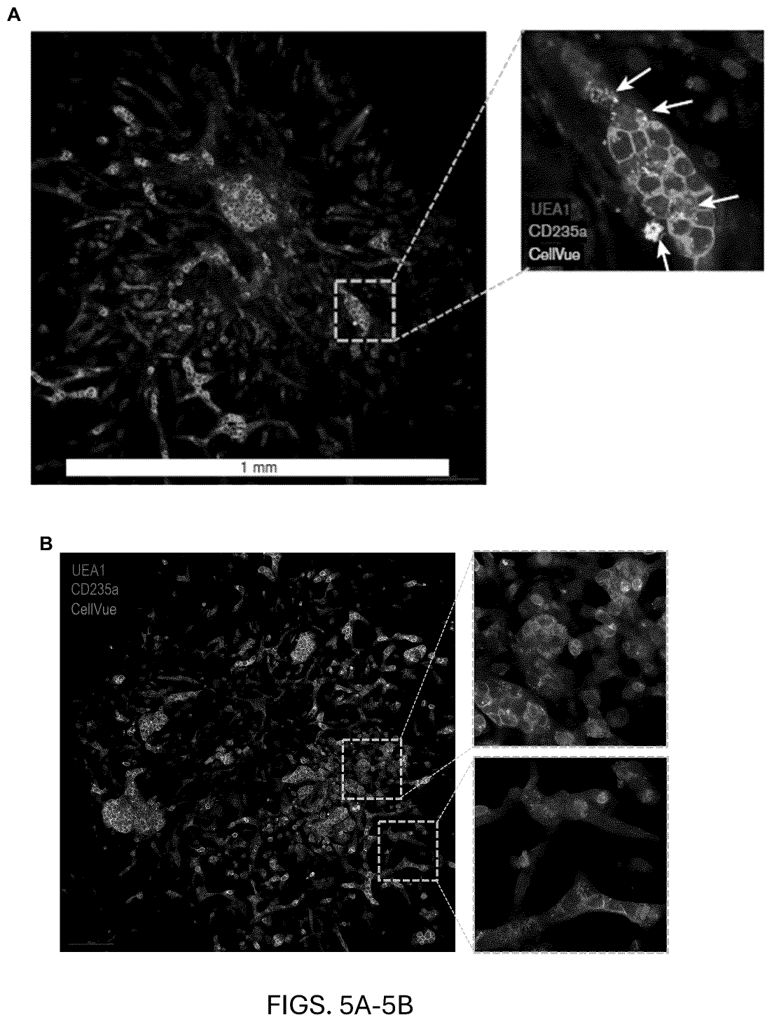

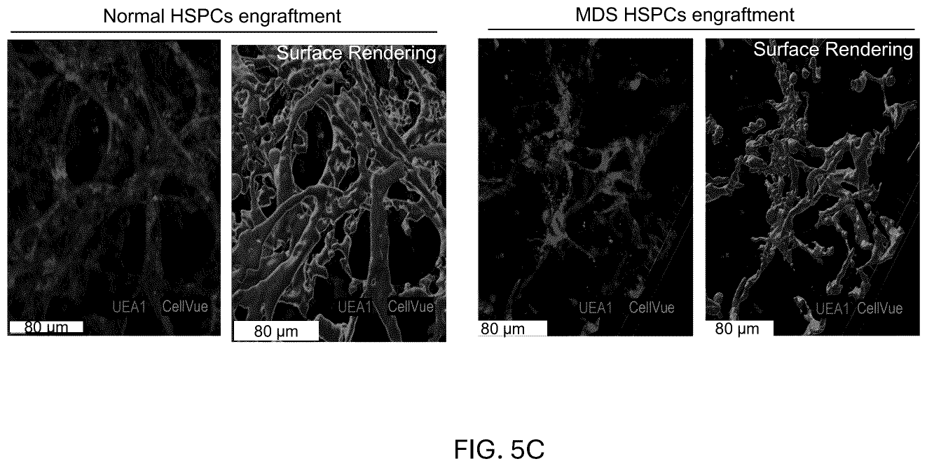

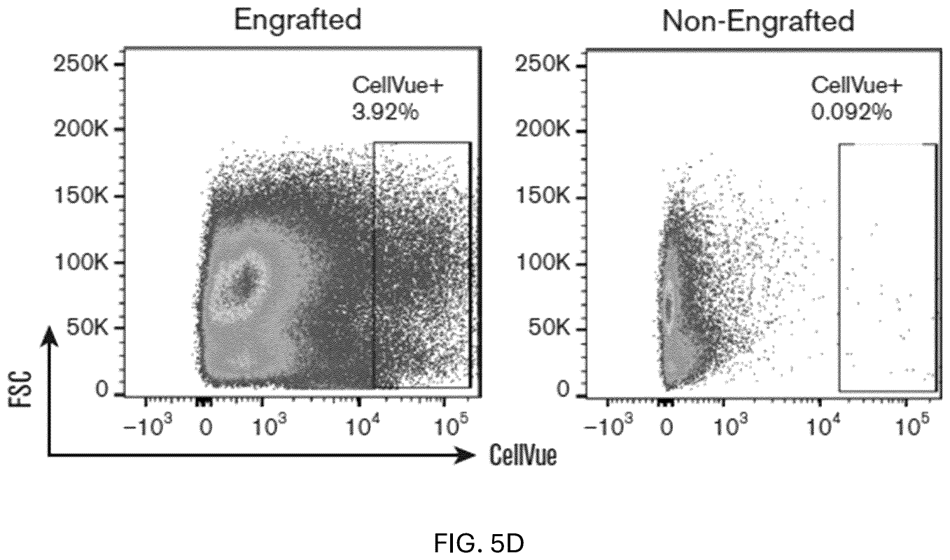

We first engrafted HSPCs derived from normal bone marrow by incubating 5×10³ CellVue-labeled CD34+ cells with each single organoid. After 72 hours, we collected the organoids, washed them vigorously to remove the non-engrafted donor cells, and performed a whole-mount imaging analysis. We found that the engrafted CellVue-positive donor cells were readily detected across the parenchyma of the bone marrow organoid. The engrafted cells were often embedded among the organoid-derived hematopoietic cells, notably in the crythroid islands (FIG. 5A). The engrafted cells could also form focal proliferative clusters, demonstrating robust fitness to the organoid niche (FIG. 5B). 3D surface rendering revealed engrafted cells within and surrounding the UEA1 positive vessels (FIG. 5C). Flow cytometry assay further confirmed the engraftment of the CellVue-labeled donor cells (FIG. 5D).

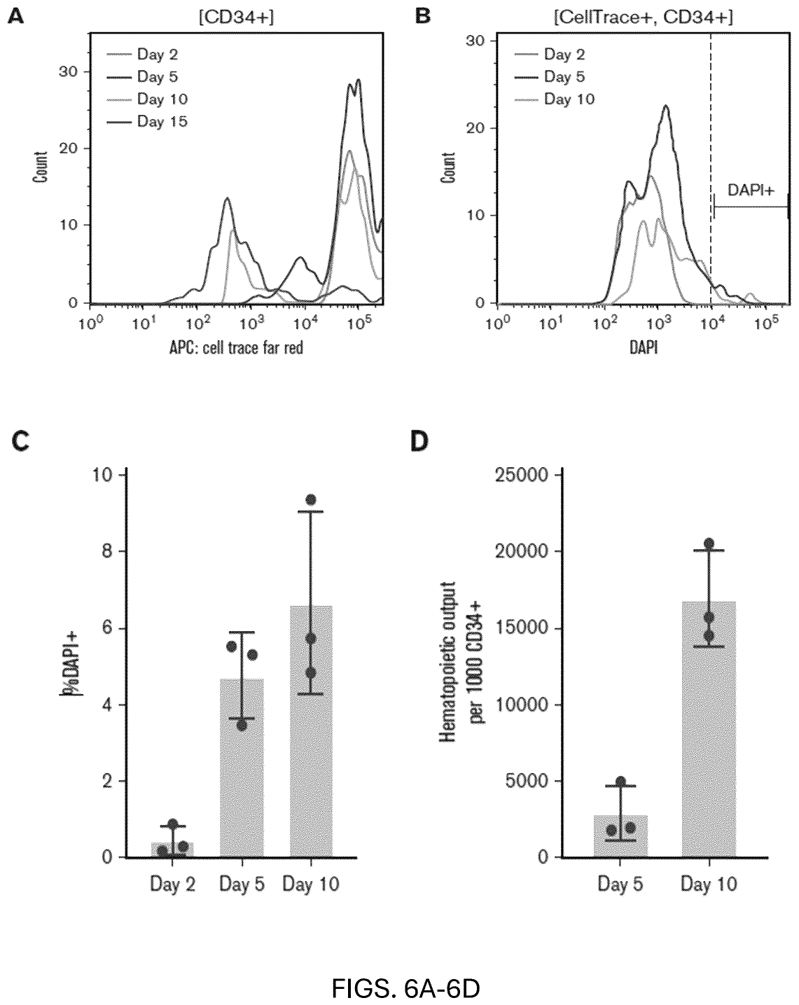

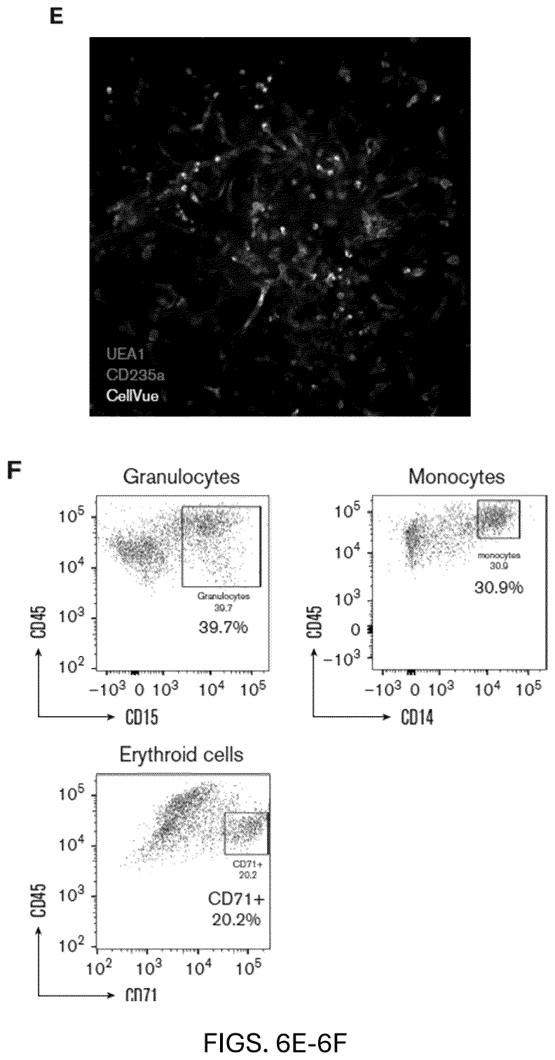

The proliferation and survival of the engrafted CD34+ HSPCs were assessed using flow cytometry. We observed a progressive reduction in the Cell Trace signal on days 5 and 10 compared to day 2, indicating that the organoid was able to maintain the self-renewal of the engrafted HSPCs (FIG. 6A). The CD34+ HSPCs engrafted in organoids showed a significant survival advantage compared to those cultured in liquid media (FIGS. 6B and 6C). Additionally, the total number of Cell Trace-positive cells increased, with approximately 16,000 hematopoictic cells generated from 1,000 engrafted CD34+ HSPCs on day 10 post-engraftment (FIG. 6D). These data demonstrate the capability of the niche microenvironment in the bone marrow organoids to support engrafted donor HSPCs and establish a foundation for using the organoid to model human hematologic diseases. We further tested the self-renewal capacity of the engrafted MDS HSPCs using a secondary engraftment assay. Indeed, CD34+ and CellTrace+ cells purified from engrafted primary organoids exhibited similar efficiencies in lodging to the appropriate niches and differentiation in different batches of organoids (FIGS. 6E-6F).

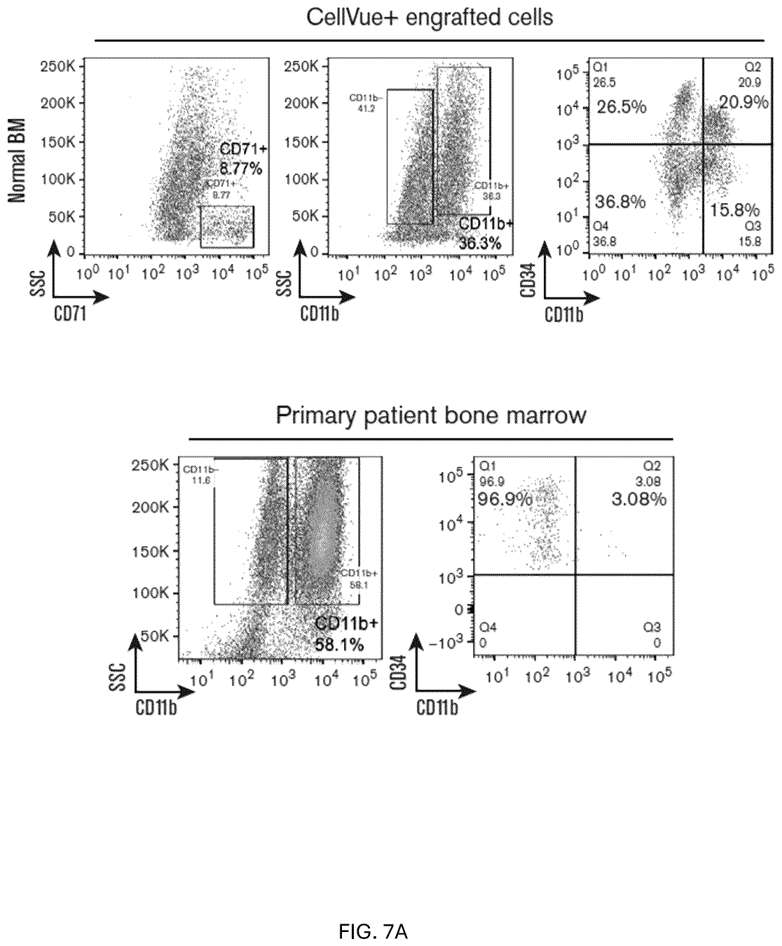

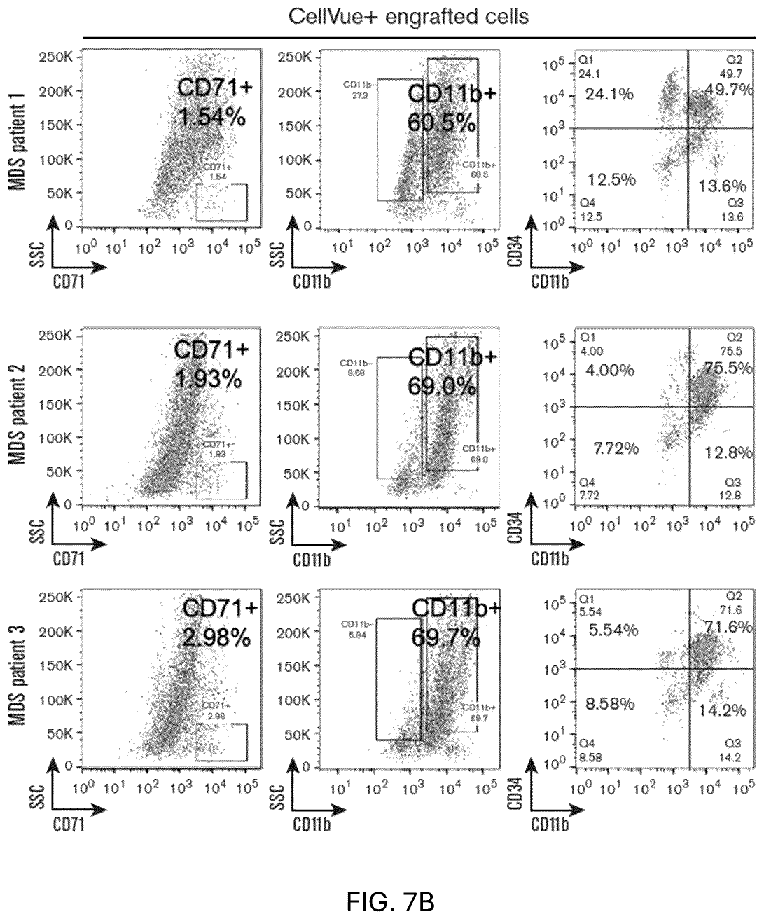

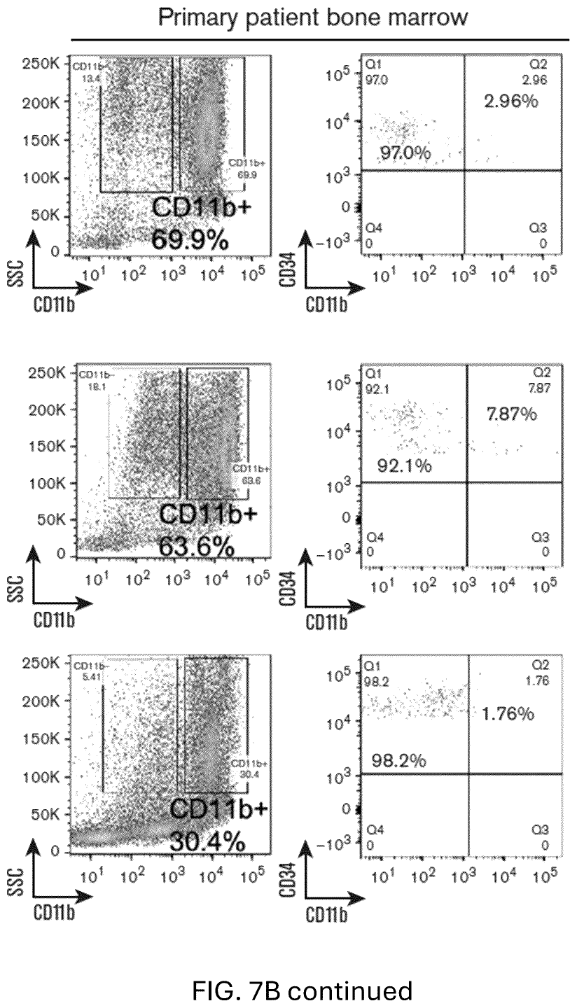

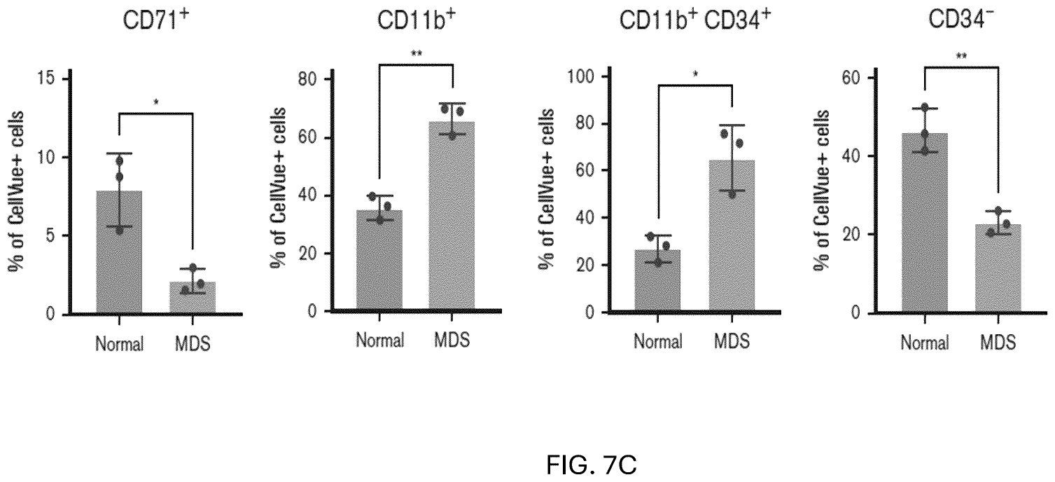

To this end, we investigated the donor HSPCs derived hematopoiesis within the bone marrow organoids using CD34+ HSPCs from patients with different subtypes of MDS and compared them to their counterparts from individuals with normal bone marrow. Flow cytometry analyses 72 hours after three independent normal HSPC engraftment assays revealed differentiation of the engrafted donor CD34+ HSPCs to CD71+ erythroid, CD11b+ myeloid cells, and downregulation of CD34 expression (FIG. 7A). Notably, a subset of CD34+ cells gained CD11b expression (27%+4.6%, mean+SD) compared to the state when they were initially purified from the primary bone marrow, indicating a possible differentiation trajectory to myeloid cells after engraftment (FIG. 7A).

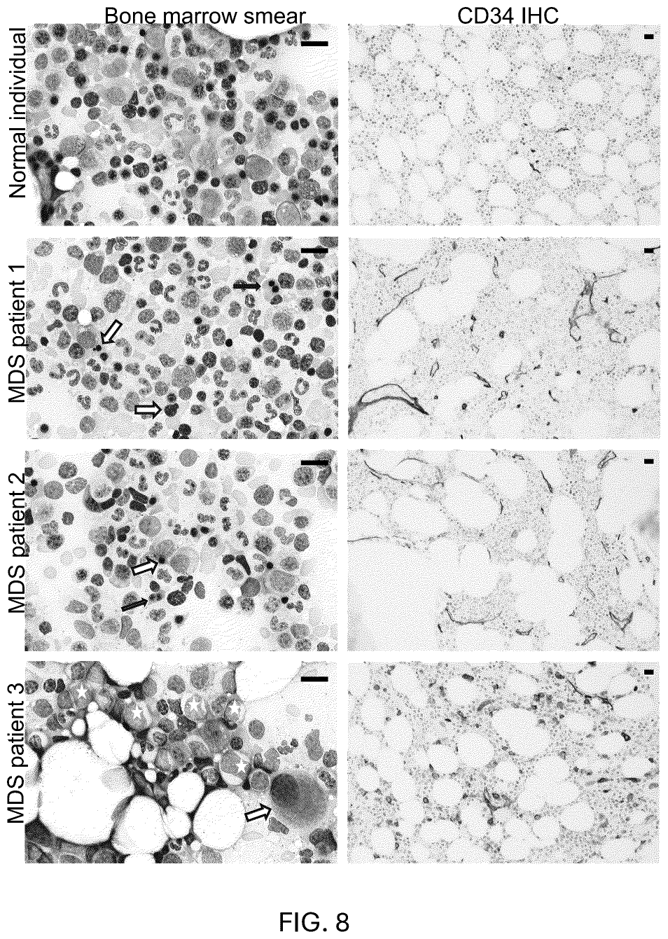

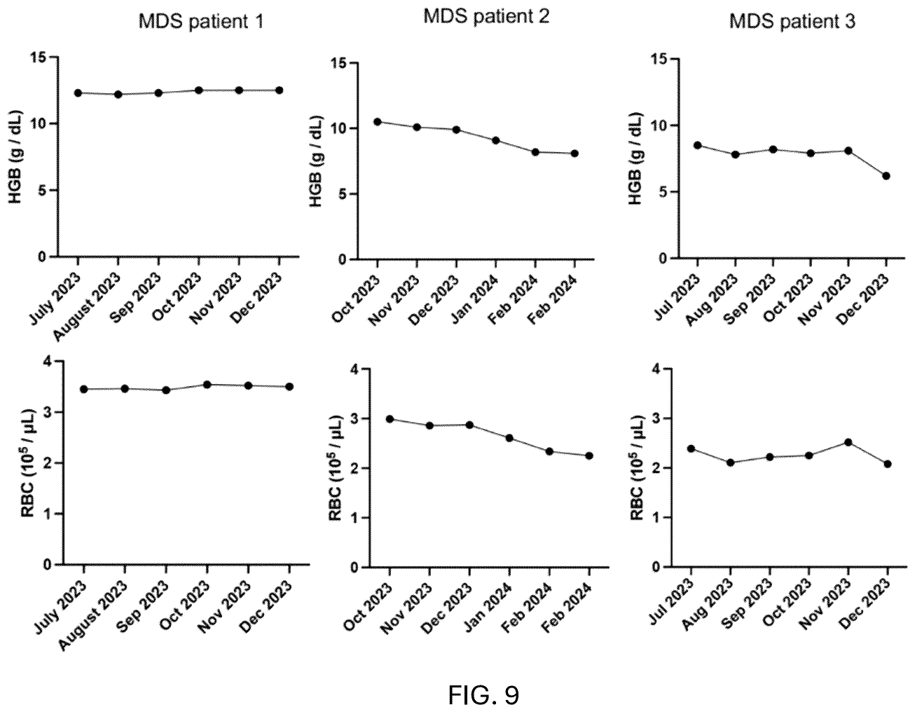

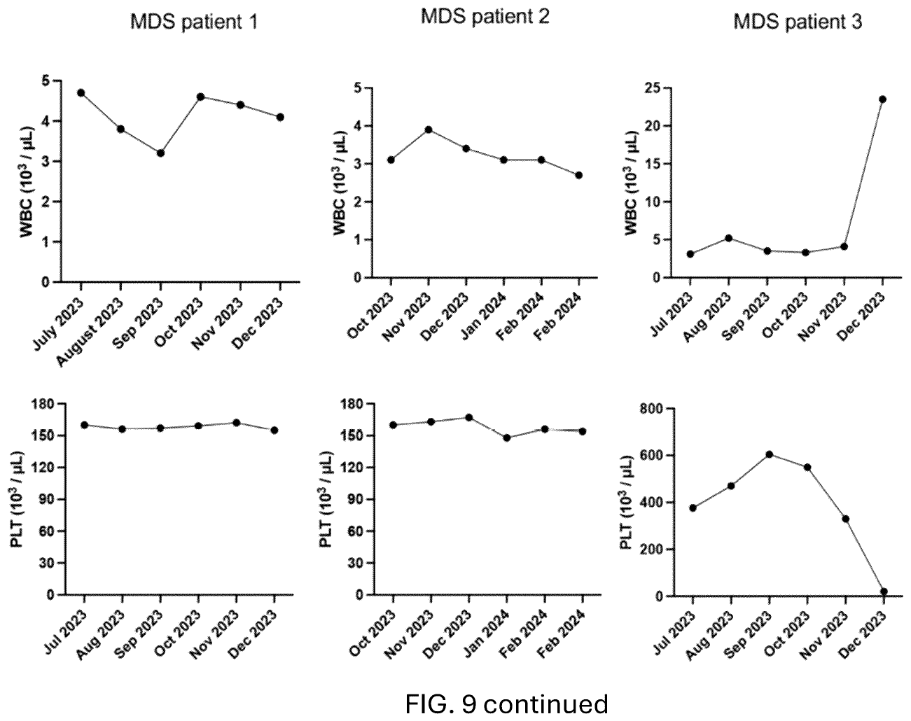

With this information, we chose 3 MDS patients with different clinical presentations to test their engraftment-derived hematopoiesis within the organoids. Patient 1 was diagnosed with MDS with low blasts based on the 5th edition of the World Health Organization (WHO) classification of hematolymphoid tumors, or MDS, not otherwise specified (NOS) with multilineage dysplasia based on the International Consensus Classification (ICC) of myeloid neoplasms. Patient 1 showed mutations in TET2, U2AF1, and DNMT3A on next-generation sequencing and trisomy 8 on cytogenetic studies. Bone marrow morphologie analyses revealed multilineage dysplasia, mainly in the erythroid and megakaryocytic lineages (FIG. 8). Clinically, patient 1 showed persistent but mild macrocytic anemia without other cytopenias (FIG. 9). Patient 2 was diagnosed with MDS with low blasts and SF3B 1 mutation based on WHO or MDS with mutated SF3B1 based on ICC. Next-generation sequencing studies showed additional ASXLI and TET2 mutations. Bone marrow biopsies showed dyserythropoiesis and frequent ring sideroblasts. Clinically, patient 2 had persistent and moderate macrocytic anemia, leukopenia, and mild thrombocytopenia (FIG. 9). Patient 3 was diagnosed with MDS with increased blasts (10-15% blasts), carrying ASXLI, KRAS, and U2AF1 mutations, as well as chromosome 5q deletion. The bone marrow biopsy showed multilineage dysplasia and frequent blasts (FIG. 8). The clinical picture of patient 3 was also the worst among all three cases and showed marked anemia, thrombocytopenia, and leukocytosis with circulating blasts (FIG. 9).

CD34+ cells derived from the MDS bone marrow samples were able to efficiently engraft to the vascular niche within the organoids, as demonstrated by confocal microscopy (FIG. 5C). Flow cytometry assays further confirm the engraftment and, more importantly, reflect the patients' clinical and pathologic presentations. The engrafted donor cells from all three MDS patients showed significantly reduced erythroid differentiation compared to the normal control (FIGS. 7B and 7C). The differentiation profiles of CD11b+ myeloid cells in the organoids correlate with those obtained from the patients' primary bone marrow flow cytometry findings. While cells from all three MDS patients showed significant retention of CD34 expression, the level of CD34 reduction, which reflects the differentiation of HSPCs, was more compromised in patients 2 and 3. This is consistent with the worse clinical presentation and cytopenias in these two patients compared to patient 1 (FIG. 7C).

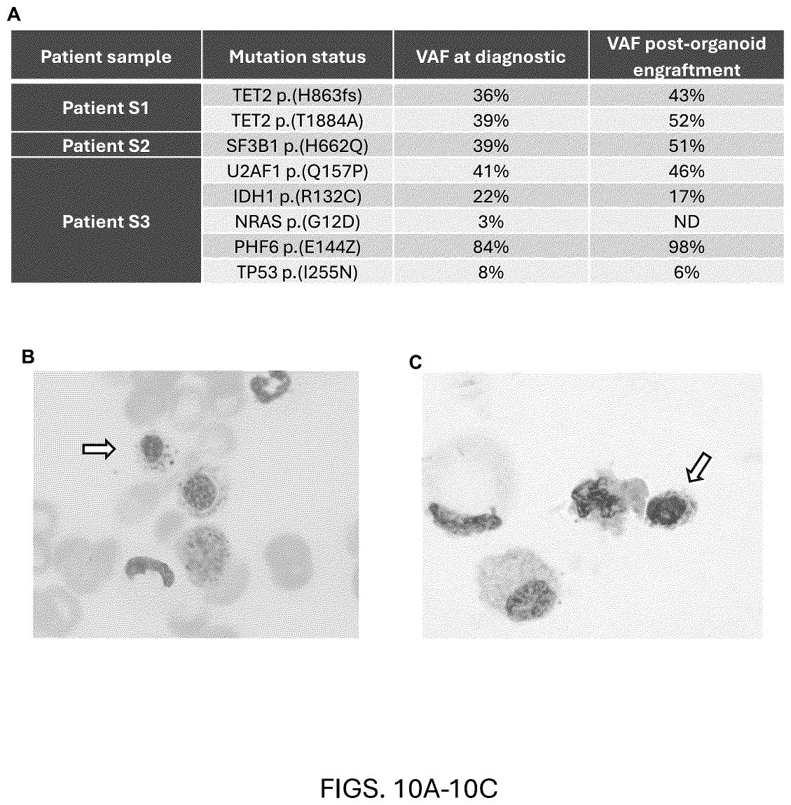

To determine if the engrafted MDS HSPCs retain their genetic mutations during proliferation and differentiation in the organoids, we engrafted CD34+ HSPCs from three independent MDS samples into the organoids. On day 10 post-engraftment, we collected Cell Trace-positive cells using FACS and conducted whole-exome DNA sequencing (WES) on the sorted cells. WES revealed that the engraftment-derived cells maintained the genetic mutations (FIG. 10A). Additionally, Patient S2, who carried the SF3B1 mutation and exhibited ring sideroblasts in the bone marrow smear (FIG. 10B), was analyzed. We digested organoids engrafted with HSPCs from Patient S2 into a single-cell suspension and performed Prussian Blue staining on the Cytospin slides, where ring sideroblasts were also found. These results indicate that the organoid can provide a microenvironment for the MDS HSPCs to maintain their pathological characteristics both genetically and morphologically.

Thus, our study introduces a replicable human bone marrow organoid model that not only is capable of autonomous hematopoiesis but also supports the growth and multi-lineage differentiation of the engrafted HSPCs. This development represents a step forward in our understanding and utilization of organoids for studying human hematopoiesis and related disease pathophysiology in a human context. The efficient and rapid engraftment of patient-derived HSPCs, especially those from MDS, is particularly important given that the field has been stagnant with ineffective engraftment of MDS cells in animal models. In addition, this technology promotes the field by providing a platform to study the ex vivo effects of different therapies for bone marrow-related diseases to predict in vivo efficacies.

### REFERENCES

1. Song Y, Rongvaux A, Taylor A, et al. A highly efficient and faithful MDS patient-derived xenotransplantation model for pre-clinical studies. Nat Commun. 2019; 10 (1): 366.
2. Lang Y, Lyu Y, Tan Y, Hu Z. Progress in construction of mouse models to investigate the pathogenesis and immune therapy of human hematological malignancy. Front Immunol. 2023; 14: 1195194.
3. Abarrategi A, Mian S A, Passaro D, Rouault-Pierre K, Grey W, Bonnet D. Modeling the human bone marrow niche in mice: From host bone marrow engraftment to bioengineering approaches. J Exp Med. 2018; 215 (3): 729-743.
4. Goyama S, Wunderlich M, Mulloy JC. Xenograft models for normal and malignant stem cells. Blood. 2015; 125 (17): 2630-2640.
5. Lysenko V, Wildner-Verhey van Wijk N, Zimmermann K, et al. Enhanced engraftment of human myelofibrosis stem and progenitor cells in MISTRG mice. Blood Adv. 2020; 4 (11): 2477-2488.
6. Martin M G, Welch J S, Uy G L, et al. Limited engraftment of low-risk myelodysplastic syndrome cells in NOD/SCID gamma-C chain knockout mice. Leukemia. 2010; 24 (9): 1662-1664.
7. Come C, Balhuizen A, Bonnet D, Porse BT. Myelodysplastic syndrome patient-derived xenografts: from no options to many. Haematologica. 2020; 105 (4): 864-869.
8. Muguruma Y, Matsushita H, Yahata T, et al. Establishment of a xenograft model of human myelodysplastic syndromes. Haematologica. 2011; 96 (4): 543-551.
9. Benito A I, Bryant E, Loken M R, et al. NOD/SCID mice transplanted with marrow from patients with myelodysplastic syndrome (MDS) show long-term propagation of normal but not clonal human precursors. Leuk Res. 2003; 27 (5): 425-436.
10. Panoskaltsis N, Mantalaris A, Wu JH. Engineering a mimicry of bone marrow tissue ex vivo. J Biosci Bioeng. 2005; 100 (1): 28-35.
11. Mantalaris A, Keng P, Bourne P, Chang A Y, Wu J H. Engineering a human bone marrow model: a case study on ex vivo erythropoiesis. Biotechnol Prog. 1998; 14 (1): 126-133.
12. Bessy T, Itkin T, Passaro D. Bioengineering the Bone Marrow Vascular Niche. Front Cell Dev Biol. 2021; 9: 645496.
13. Bourgine P E, Klein T, Paczulla A M, et al. In vitro biomimetic engineering of a human hematopoietic niche with functional properties. Proc Natl Acad Sci USA. 2018; 115 (25): E5688-E5695.
14. de Janon A, Mantalaris A, Panoskaltsis N. Three-Dimensional Human Bone Marrow Organoids for the Study and Application of Normal and Abnormal Hematoimmunopoiesis. J Immunol. 2023; 210 (7): 895-904.
15. Frenz-Wiessner S, Fairley SD, Buser M, et al. Generation of complex bone marrow organoids from human induced pluripotent stem cells. Nat Methods. 2024.
16. Khan A O, Rodriguez-Romera A, Reyat J S, et al. Human Bone Marrow Organoids for Disease Modeling, Discovery, and Validation of Therapeutic Targets in Hematologic Malignancies. Cancer Discov. 2023; 13 (2): 364-385.
17. Han X, Mei Y, Mishra R K, et al. Targeting pleckstrin-2-Akt signaling reduces proliferation in myeloproliferative neoplasm models. J Clin Invest. 2023.
18. Mei Y, Ren K, Liu Y, et al. Bone marrow-confined IL-6 signaling mediates the progression of myelodysplastic syndromes to acute myeloid leukemia. J Clin Invest. 2022; 132 (17).
19. Liu Y, Mei Y, Han X, et al. Membrane skeleton modulates erythroid proteome remodeling and organelle clearance. Blood. 2021; 137 (3): 398-409.
20. Zhao B, Liu H, Mei Y, et al. Disruption of erythroid nuclear opening and histone release in myelodysplastic syndromes. Cancer Med. 2019; 8 (3): 1169-1174.
21. Zhao B, Mei Y, Cao L, et al. Loss of pleckstrin-2 reverts lethality and vascular occlusions in JAK2V617F-positive myeloproliferative neoplasms. J Clin Invest. 2018; 128 (1): 125-140.

### Example 2

Predicting clinical drug response using the bone marrow organoid engraftment model

To investigate whether the bone marrow organoid engraftment model can be used for drug sensitivity testing, we engrafted CD34+ cells from three different acute myeloid leukemia (AML) patients into normal human bone marrow organoids using the method described previously (1).

Bone marrow samples were collected from patients diagnosed with AML who were scheduled to initiate their first cycle of treatment with azacitidine plus venetoclax (Aza+Ven). Inclusion criteria required patients to be newly diagnosed or Aza+Ven−treatment-naïve and eligible for venetoclax-based therapy.

Clinical response (Table 1) to Aza+Ven therapy was retrospectively assessed based on documented bone marrow biopsy results following the first treatment cycle. The response was assessed by reviewing the blast percentage reported in the clinical notes. Responses were categorized as follows:

TABLE 1

| Patient | Diagnosis | Drug | Result | Notes |
|---|---|---|---|---|
| #1 | Relapsed AML | Azacitidine + | Partial | Blasts |
| | | Venetoclax | response | dropped |
| | | | during | from 80% |
| | | | cycle 1; | to ~20% |
| | | | Resistant | (cycle 1) |
| | | | during | but rose |
| | | | cycle 2 | to 30% |
| | | | | (cycle 2) |
| #2 | Secondary AML | Azacitidine + | Response | No |
| | from | Venetoclax | | detectable |
| | myeloproliferative | | | blasts after |
| | neoplasm (MPN) | | | cycle 1 |
| #3 | Newly diagnosed | Azacitidine + | Resistant | Persistent |
| | AML | Venetoclax + | | blasts at |
| | | IMGN632 (an | | cycle 1 day |
| | | investigational | | 20, and |
| | | CD123- | | cycle 2 and |
| | | targeting | | 22 |
| | | antibody-drug | | |
| | | conjugate) | | |

Response: Patients with a decrease in marrow blasts to <5%.

Partial response: Patients with blasts between 5% and 20%.

Resistant: Unchanged or increased blast percentages compared to the beginning of the cycle.

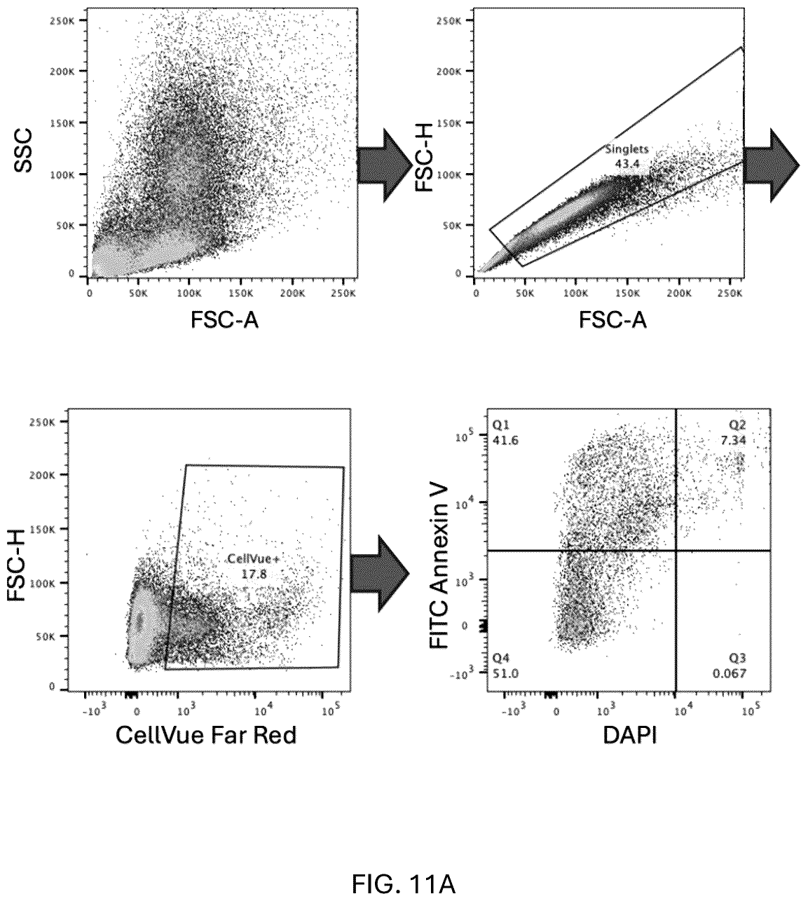

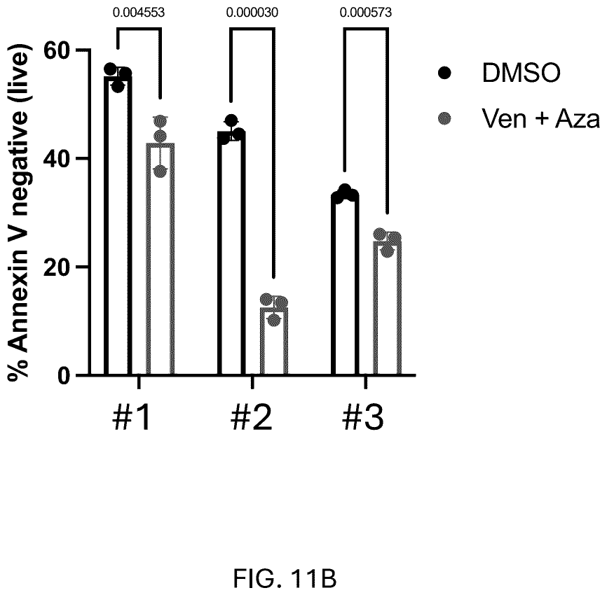

After 48 hours of engraftment, the engrafted organoids were treated with the combination of 1 μM azacitidine and 200 nM venetoclax for 48 hours. 0.1% DMSO was used as a control. Subsequently, two engrafted organoids were combined into a single tube as a sample, and three samples were collected for each group to ensure biological replication. The engrafted organoid samples were dissociated in collagenase at 37° C. for 60 minutes, then stained with FITC-conjugated Annexin V and DAPI for flow cytometry analysis. The percentage of Annexin V and DAPI double-negative cells was used to reflect cell viability (FIG. 11A).

Flow cytometry analyses (FIG. 11B) of the engrafted organoids revealed that treatment with Aza+Ven significantly reduced the percentage of viable engrafted cells across all three patient-derived samples. Notably, the magnitude of viability reduction correlated with the clinical drug response. The sample derived from patient FA, who demonstrated a strong clinical response, exhibited the most pronounced decrease in viability, with a 72.2% reduction compared to the DMSO-treated control. In contrast, samples derived from patients GN and PM, who exhibited no clinical response following cycles one and two, respectively, showed only modest reductions in viability, with decreases of 25.85% and 22.3% compared to DMSO controls. These results indicate that the bone marrow organoid model may serve as a predictive platform for assessing individual patient sensitivity to Aza+Ven therapy ex vivo, potentially mirroring in vivo clinical outcomes. Compared to traditional liquid culture systems, which typically require ˜7×10⁵ cells per assay (2), the bone marrow organoid engraftment model requires significantly fewer patient-derived CD34+ cells for drug sensitivity testing—as few as 6×10⁴ cells per assay-making it particularly advantageous in settings with limited sample availability, such as minimal residual disease (MRD). In addition to reduced cell input, this model preserves critical aspects of the bone marrow microenvironment, including three-dimensional architecture, stromal interactions, and extracellular matrix components, which are absent in liquid culture. These features allow for more physiologically relevant assessment of drug response, particularly for therapies whose efficacy depends on niche-mediated survival signals.

### REFERENCES

1. K. Ren et al., Development of iPSC-derived human bone marrow organoid for autonomous hematopoiesis and patient-derived HSPC engraftment. Blood Adv 9, 54-65 (2025).
2. S. Kytölä et al., Ex vivo venetoclax sensitivity predicts clinical response in acute myeloid leukemia in the prospective VenEx trial. Blood 145.4 (2025): 409-421.

What is claimed:

1. A method for engrafting a bone marrow organoid with CD34+ hematopoietic stem and progenitor cells (HSPCs), the method comprising:
(a) incubating the organoid in a cell suspension comprising CD34+ HSPCs in a serum-free stem cell culture medium and a basement membrane matrix;
(b) adding to the organoid an engraftment-promoting medium comprising the serum-free stem cell culture medium, stem cell factor (SCF), fms related receptor tyrosine kinase 3 (FLT3), thrombopoietin (TPO), erythropoietin (EPO), and interleukin-3 (IL-3).

2. The method of claim 1, wherein the step of incubating the organoid in the cell suspension is done for between about 15 and about 30 minutes.

3. The method of claim 1, wherein the step of incubating the organoid in the cell suspension is done at between about 36.5 and about 37.5° C.

4. The method of claim 1, wherein the engraftment-promoting medium comprises each of SCF, FLT3, TPO, EPO, and IL-3 at between about 5 and about 15 ng/mL.

5. The method of claim 4, wherein the engraftment-promoting medium comprises each of SCF, FLT3, TPO, EPO, and IL-3 at about 10 ng/mL.

6. The method of claim 1, wherein the cell suspension comprises between about 1×10³ and about 10×10³ HPSCs.

7. The method of claim 6, wherein the cell suspension comprises about 5×10³ HSPCs.

8. The method of claim 1, wherein the HSPCs are human HSPCs.

9. The method of claim 1, wherein the HSPCs are isolated from a bone marrow sample.

10. The method of claim 1, further comprising preparing the cell suspension before step (a), wherein preparing the cell suspension comprises: suspending the HSPCs in serum-free stem cell culture medium; and adding basement membrane matrix, wherein the basement membrane matrix is at about 4° C.

11. The method of claim 1, wherein before step (a), the organoid is matured for between about 18 and about 24 days.

12. The method of claim 11, wherein the organoid is matured for about 21 days.

13. The method of claim 1, further comprising generating the organoid before step (a), wherein generating the organoid comprises:
culturing induced pluripotent stem cells (iPSCs) on the basement membrane matrix;
disrupting the cells to form aggregates;
plating the aggregates onto an ultra-low attachment substrate to form embryo bodies;
contacting the embryo bodies with bone morphogenic protein-4 (BMP4), vascular endothelial growth factor A (VEGFA), fibroblast growth factor-2 (FGF2), and
interleukin-21 (IL-21) under hypoxia conditions;
culturing the embryo bodies with SCF and FLT3 under normoxia;
incubating the embryo bodies in a hydrogel;
adding a stem cell differentiation medium supplemented with VEGFA, vascular endothelial growth factor C (VEGFC), FGF2, BMP4, FLT3, SCF, granulocytic colony-stimulating factor (G-CSF), TPO, EPO, IL-3, and interleukin-6 (IL-6) to the hydrogel form organoids;
seeding the organoids individually in an ultra-low attachment plate in the stem cell differentiation medium supplemented with FGF2, SCF, FLT3, IL-3, TPO and EPO.

14. The method of claim 13, wherein the iPSCs are human iPSCs.

15. A composition comprising a serum-free cell culture medium, SCF, FLT3, TPO, EPO, and IL-3.

16. The composition of claim 15, wherein each of the SCF, FLT3, TPO, EPO, and IL-3 are at between about 5 and 15 ng/mL.

17. A kit comprising the composition of claim 15; and at least one of a CD34+ HSPC and a bone marrow organoid.

18. A kit comprising SCF, FLT3, TPO, EPO, and IL-3 in separate containers.

19. The kit of claim 18, further comprising at least one of a CD34+ HSPC and a bone marrow organoid.

20. A method for predicting drug response in a subject having a myeloid disease, the method comprising:
(a) engrafting a bone marrow organoid with CD34+ HSPC cells isolated from the subject by the method of claim 1, thereby producing an engrafted organoid;
(b) treating the engrafted organoid with the drug; and
(c) assessing viability of the CD34+ HSPC cells in the engrafted organoid.
